(19) United States
(12) Patent Application Publication
(10) Pub. No.: US 2019/0099508 A1
(43) Pub. Date: Apr. 4, 2019

(54) HYPER TUMBLER

(71) Applicant: Hyper Light Technologies, LLC, Nashville, NC (US)

(72) Inventor: Kurt A. Garrett, Raleigh, NC (US)

(73) Assignee: Hyper Light Technologies, LLC, Nashville, NC (US)

(21) Appl. No.: 15/720,295

(22) Filed: Sep. 29, 2017

(51) Int. Cl. A61L 2/10; A61L 2/26

(52) U.S. Cl. CPC A61L 2/10; A61L 2202/121; A61L 2202/11; A61L 2/26

(57) ABSTRACT

The device is a tumbler which provides a high energy UV light with low or no heat suitable for disinfecting objects, especially heat sensitive objects.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a medical sterilization appliance. In particular, it relates to a medical sterilization appliance utilizing high intensity UV light wherein essentially, or mostly, all the heat has been removed from the system.

### Description of Related Art

The microbiological sterilization of various objects has been pivotal in the production in use of medical products as well as items of everyday use. Various technologies have been developed to achieve this sterilization, including UV-irradiation, gamma-ray irradiation (or gamma irradiation), chemical sterilization, heat sterilization, autoclaving, and ultrafiltration. Because these technologies destroy microorganisms, they are inherently damaging to other biological components that may be in the product to be sterilized. In addition, the heat generated by some of these technologies can damage plastics or other heat sensitive objects. In light of this fact, a particular technology may not always be acceptable for sterilizing a given product. Of the sterilizing technologies available, most are not acceptable for these applications. Chemical sterilization, heat sterilization, and autoclaving all damage or alter many materials, destroying the materials or the device it is used on. The prior processes effectively kill the microbe that utilizes these molecules for life processes. However, this inactivation of biological molecules that occurs with prior art technologies is inherently problematic.

Ultrafiltration, a recent technology relative to the others mentioned here, requires the use of filters with a very minute pore size (at least <0.45 microns). These filters are an inherently slow means of sterilization and may not be suitable for solutions of high viscosity or solutions that contain desirable particles, such as cells, that are larger than the pore diameter and, consequently, too large to pass through the filter and inadequate for solid objects. Gamma-irradiation is a technology not commonly used for microbial sterilization, although it can be used to ensure the sterility of the majority of, if not all, biotechnology products. One major reason for its lack of widespread use for microbial sterilization is that it utilizes a radiation source, such as radioactive cobalt, that is very radioactive, and thus, very dangerous. This technology requires extensive shielding and control systems to prevent accidental exposure to operators and others. These protective requirements are economically expensive, often prohibitively so. Therefore, gamma-irradiation is often not an economically acceptable technology or a safe technology for sterilization of biotechnology products. Additionally, gamma-irradiation sterilizes products by lysising the biological molecules contained in microorganisms. This photochemical mechanism of sterilization may also degrade the desired product, rendering it inactive, and thus defeating the purpose of the sterilization.

UV-irradiation has been used extensively for microbial sterilization. UV light breaks the hydrogen bonds between adenine-thymine moieties in the DNA polymer that comprises the genome of the cell or virus and catalyzes the formation of a cyclobutane dimer between adjacent thymine moieties. This disruption of the genome blocks the replication cycle of the cell or virus, effectively inhibiting growth of the organism.

Generally, devices that use UV light to sterilize products are composed of a power supply (ballast), a UV light source, a light-focusing and/or light-conducting device, a light filter, and a control system to assure proper operation. The ballast is designed to supply power to the lamp in a reliable fashion in order to ensure continuous optimal function of the lamp. A variety of UV light sources exist and are known in the prior art, including pulsed, gas-filled flash lamps, spark-gap discharged apparatus, or low-pressure mercury vapor lamps. Traditionally, low-pressure mercury vapor lamps have been used for microbial sterilization devices because these lamps are relatively inexpensive to operate and emit relatively higher amounts of UV-irradiation than other sources. Other types of vapor lamps are also used, including mercury-xenon (HgXe) lamps. In particular, a preferred embodiment, according to the present invention, employs a pencil type Hg(Ar) spectral calibration lamp. These lamps are compact and offer narrow, intense emissions. Their average intensity is constant and reproducible. They have a longer life relative to other high wattage lamps. Hg(Ar) lamps of this type are generally insensitive to temperature and require only a two-minute warm-up for the mercury vapor to dominate the discharge, then 30 minutes for complete stabilization. Recent developments have provided safer UV light production, reducing heat, but the application of such technology is still in the developmental stage.

By way of background, light is conventionally divided into infrared light (780 nm to 2600 nm), visible light (380 nm to 780 nm), near UV light (300 nm to 380 nm), and far UV light (170 nm to 300 nm). Most UV lamp sources emit light at discrete wavelengths and include filters to filter out or block wavelengths other than the specific UV wavelength, especially 254 nm. In the UV region, the most notable UV emission occurs at 254 nm. It is known that mercury vapor lamps emit radiation at 254 nm. This wavelength can damage the genome of cells and viruses, thus inhibiting their replication, thereby sterilizing the cells and viruses. Therefore, generally in the prior art, a single wavelength detector, tuned to 254 nm, has been used to determine the amount of UV radiation reaching the target. In order to optimize the UV light output efficiency of the lamp source, at least one filter was interposed in the light path in order to block non-UV light from reaching the target, allowing only UV and proximate UV light to reach the target. Therefore, the industry has evolved over time with the solidly established paradigm that 254 nm is the sole and exclusive wavelength of importance for UV sterilization. As such, the prior art teaches away from the inclusion of non-UV wavelength light for microbial sterilization apparatus. Furthermore, this paradigm not only teaches that polychromatic or broad spectrum light as irrelevant or unimportant, but disadvantageous.

In sharp contrast to UV irradiation which utilizes a photo thermal and/or photochemical mechanism, Dunn (U.S. Pat. No. 4,871,559; issued Oct. 3, 1989 to Dunn et al., titled METHODS FOR PRESERVATION OF FOODSTUFFS) teaches that the inactivation of enzymes by visible and infrared radiation utilizes a photo thermal mechanism. When applied at high intensity and in combination, UV, IR, and visible light, which are components included in a complete spectrum, result in significant shelf life and stability enhancements of food products by the killing of microbes and by the inactivation of degradatory enzymes. Notably, the prior art for UV sterilization in biotechnology applications teaches away from Dunn's approach to multiple component light application since the prior art teaches that filtered UV light is desirable while nonfiltered UV light is undesirable for sterilization of microorganisms. Prior art teaches away from the use of non-filtered UV light for the sterilization of microorganisms. Disadvantageously, the activities of biotechnology products are frequently based on enzymatic activity or require the tertiary or quaternary structure of proteins for activity. Therefore, sterilization techniques, like Dunn, that indiscriminately degrade proteins and enzymes in the process of sterilization, are not acceptable for use with biotechnology products. Thus, there remains a need for a sterilization appliance device that is easy to use for solid and liquid subjects that can effectively sterilize without damaging the products so treated. The types of objects for such sterilization could include medical devices, toys and other objects in a children setting, veterinarian and medical offices, and the like.

Therefore, there remains a need not solved by the prior art to more effectively sterilize objects.

### BRIEF SUMMARY OF THE INVENTION

The present invention relates to a high UV output light which utilizes a dichroic filter to remove a substantial amount, or all, of heat from the UV source mounted in a sealable container with a tumbler type action that solves the problems posed above and also provides other benefits, as will be obvious to the reader.

Accordingly, in one embodiment, there is a sterilization appliance cabinet for sterilizing an object comprising:
a) a sealing door;
b) a cabinet interior having UV reflective walls and a ventilated tumbler for placement of the object;
c) a lamp assembly comprising a low heat, high energy UV lamp;
d) an appliance control panel; and
e) wherein the lamp assembly is positioned to deliver sufficient low heat, high intensity UV light to the cabinet interior tumbler to sterilize the object.

In another embodiment, there is a method of sterilizing an object comprising:
a) placing the object to be sterilized in a tumbler of a sterilization appliance cabinet, the cabinet comprising:
   i. a sealing door;
   ii. a cabinet interior having reflective walls and a ventilated tumbler;
   iii. a lamp assembly comprising a low heat, high energy UV lamp;
   iv. a UV transparent tray designed to hold the object in the cabinet interior;
   v. an appliance control panel; and
   vi. wherein the lamp assembly is positioned to deliver a low heat UV polychromatic light to the cabinet interior; and
b) operating the appliance for a time sufficient to sterilize the object placed in the cabinet.

### BRIEF DESCRIPTION OF THE DRAWINGS

FIG. 1 is a schematic view of the sterilization tumbler.

FIG. 2 is an embodiment of the sterilization tumbler.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “sterilization appliance cabinet” refers to a device for the specific purpose of placing an object inside the cabinet such as a medical device, toy, or other object for the purpose of sterilizing it. An advantage of the present cabinet is there is very little heat involved in the process. It is also a safety advantage that the cabinet is sealed from the environment and thus the user during the sterilization process. The tumbling effect produced by the cabinet improves the exposure of the object to the UV light and thus decreases the time to sterilize.

As used herein, the term “sterilizing” refers to a device that has a light source producing a spectrum of light capable of killing a microorganism, such as a bacteria or virus that is on a solid (substrate). In particular, it produces a wide UV spectrum (i.e. more than just an isolated wavelength) or narrow, even though it can produce other spectrums of light and, in one embodiment, the light produces a high UV output. Solid substrates refer to non-gas or non-liquid substrates, such as metals, plastics, organic material, and the like, including inanimate solids. Since the present invention removes heat, narrow or single spectrum light can be utilized as well as the broad spectrum of light.

As used herein, the term “object” refers to those items one would put into the cabinet tumbler for sterilization. Virtually anything that will fit into the cabinet tumbler interior can be sterilized. This includes medical, dental, veterinarian devices, objects found in office work areas, and childcare toys and other objects in the daycare area, as long as not sensitive to being tumbled.

As used herein, the term “lamp assembly” refers to a lamp, an optical device including a dichroic reflector as well as the holder for the lamp assembly in the cabinet, vents in the top of the cabinet, and anything else associated with the use of the lamp. The term “optical device” refers to a device that collects light reflected off of the dichroic reflector and focuses the light into a high output stream. The focusing creates a high intensity light output. The device can be electric powered or have a manual way to focus the light. US Patent publication number US2017/0028089 published on Feb. 2, 2017 in the name of Kurt A. Garrett and U.S. patent application Ser. No. 15/712,559 filed on Sep. 22, 2017 in the name of Kurt A. Garrett describe the parts of the lamp and function of the low heat sterilization lamp in further detail and is incorporated by reference herein.

As used herein, the term “dichroic reflector” refers to any of a dichroic focus, reflector, mirror, lens or the like that takes light from the light source and allows some or all of the thermal energy to pass through the reflector while taking the light, especially the UV light, to be reflected for focusing. In one embodiment, there may be more than one dichroic reflector but at least one must focus the light for use. The dichroic reflector can be any shape that works to either remove heat or focus the light but, in one embodiment, it is an elliptical shape for focusing. In one embodiment, an elliptical dichroic reflector is used with an arc lamp. This is different from a dichroic filter which only filters or reflects light but does not pass heat wavelengths through it. the dichroic filter can be a powered or unpowered device.

As used herein, the term “lamp” refers to a light output of about at least 80 lumens per watt output. In order to achieve this high intensity light output, one cannot use low or medium pressure lamps that produce UV light, as they do not produce enough UV light output. An arc discharge lamp does not produce the level of light output intensity needed. In order to achieve the high intensity output needed, one can add to the arc discharge lamp's light output an elliptical reflector which collimates the polychromatic (or narrower length) light into still greater intensity (intensity being understood as energy per area) of about 100 lumens per watt (i.e. producing the high intensity light output needed).

As used herein, the term “power supply” refers to an AC or DC source that powers the light supply and, where needed, the optical device or any other part of the device.

As used herein, the term “polychromatic” refers to a light comprising multiple wavelengths of light. It is clear that the present invention works with any high energy light source of UV light.

As used herein, the term “predetermined exposure period” refers to the time period that light produced by the device is shown on a microorganism in order to kill it. In one embodiment, it is from about 0.01 seconds to about 5 seconds. In one embodiment, a shutter is utilized to open, close, and modulate the passage of light from the light source to the microorganism. The time will be affected by a number of things including the light intensity, the tumbling and its speed, and the like but is well within the scope of one skilled in the art.

As used herein, the term “sealing door” refers to a door which isolates the UV light from the lamp assembly from the user, thus eliminating any concerns or dangers of human exposure to the UV light utilized in the present invention. There can be a viewing window in the sealing door (or anywhere in the cabinet), and the door can be designed such that it will not open anytime the device is being operated.

As used herein, the term “cabinet tumbler interior” refers to the inside of the tumbler, including the walls, ceiling, and floor of the tumbler, wherein the tumbler, in one embodiment, is a cylindrical shape, as shown in the Figures, with holes to allow light to penetrate into the tumbler. The interior of the cabinet is sealed from the outside, including from the user of the appliance. The interior of the tumbler and, in one embodiment, the rest of the interior of the device is covered (walls, ceiling, and floor) with reflective activation materials. In other words, material that is highly reflective of the UV light is being utilized in the sterilization process.

As used herein, the term “appliance control panel” refers to user controls on the outside of the appliance that can turn the appliance on and off, set times and/or intensities, lights, timers, tumbler speed, and the like.

As used herein, the term “positioned” refers to the light from the lamp assembly being focused such that any object in the cabinet tumbler interior receives a sufficient dose of UV light for sterilization through the holes in the walls of the tumbler or because the tumbler is UV transparent.

The present invention relates, generally, to microbial sterilization (or DNA disruption, DNA inactivation) and, more particularly, to microbial sterilization using brief pulses of high-intensity polychromatic light or narrowed wavelength light directed inside a cabinet from a light assembly. One of the objects of the present invention is to improve on the prior art by more effectively sterilizing objects, as defined above, especially those that are heat sensitive (e.g. sterilizing a medical device or other object comprising heat sensitive plastic). A further object of the present invention is the use of a shutter mechanism for the modulation of the exposure period to polychromatic, full spectrum light. A further object of the present invention is the use of a dichroic reflector for removal of thermal energy and the focusing (concentrating) of polychromatic, full spectrum light. A further object of the present invention is the use of an electronic circuit board for modulating lamp power, thermals, and shutter timer of polychromatic, full spectrum light or the narrower spectrum light.

Thus, it is one aspect of the present invention to provide an appliance that will sterilize objects placed in it, especially when those objects are heat sensitive, by way of high-intensity polychromatic or broad spectrum light irradiation, including UV-irradiation. It is another aspect of the present invention to provide an apparatus using light that has been filtered through a fluid to absorb the infrared region of the light spectrum in order to minimize the photo thermal damage of the irradiated object.

### DRAWINGS

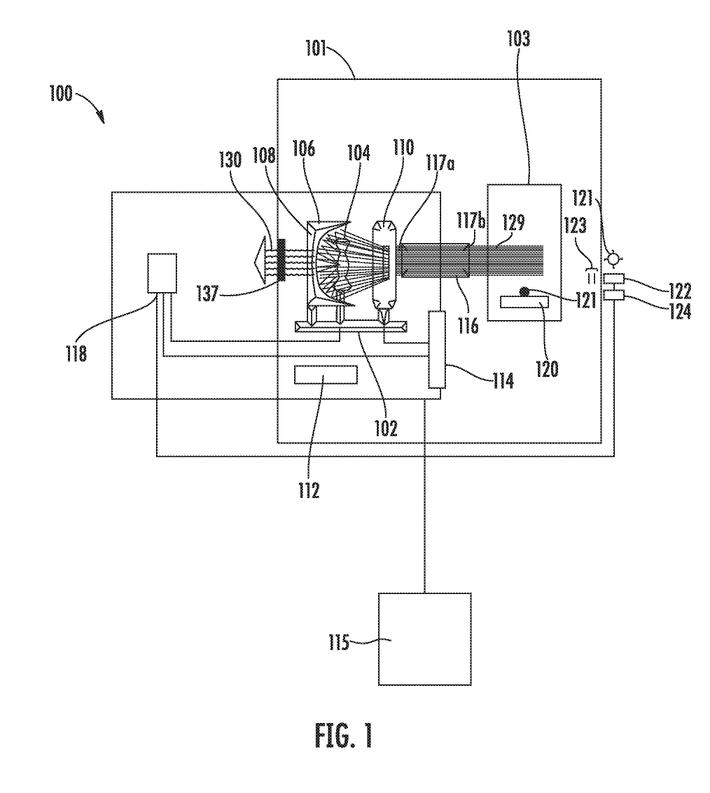

Now referring to the drawings, an embodiment of the invention provides an appliance for microbial sterilization of items placed in a tumbler inside a cabinet. It is shown generally schematically 100, as shown in FIG. 1. FIG. 1 shows that the device for microbial sterilization 100 can include: cabinet 101, a power supply 102, tumbler 103, the lamp assembly including a UV light source 104, at least one optical device 106 (which, in this embodiment, includes a dichroic reflector 108), a light shutter mechanism 110, a cooling fan 112, a timer 114 which is controlled by control panel 115, a light guide or light guide-conducting device 116 (that can also function as an infrared light filter) with a first end 117a and a second end 117b, and an exposure control system 118 to assure proper operation. Also featured are UV-sensitive diodes in a light-screened box 122, a detector circuit 124, multiplexer(s), coated mirrors as well as a UV-selective filter 123. FIG. 1 also shows the invisible infrared light (radiated heat) 130 escaping through cabinet vent 137, a beam of incident light 129, a microorganism 121, and an object 120 in the tumbler 103. The components of the embodiment are configured, positioned, and connected such that the power supply 102, in this embodiment, consisting of an electronic circuit board, provides energy to the system. In particular, the power supply provides energy to the UV light source 104, which emits a light that is reflected off the at least one optical device 106, and otherwise focused or directed into the light guide 116. The dichroic reflector 108 provides a means for removing heat from the system. The cooling fan 112 provides another means for removing excess heat from the system. The shutter mechanism 110, timer 114, and control system 118 are interconnected to provide a controlled on/off light output which reaches the object 120 having microorganisms 121.

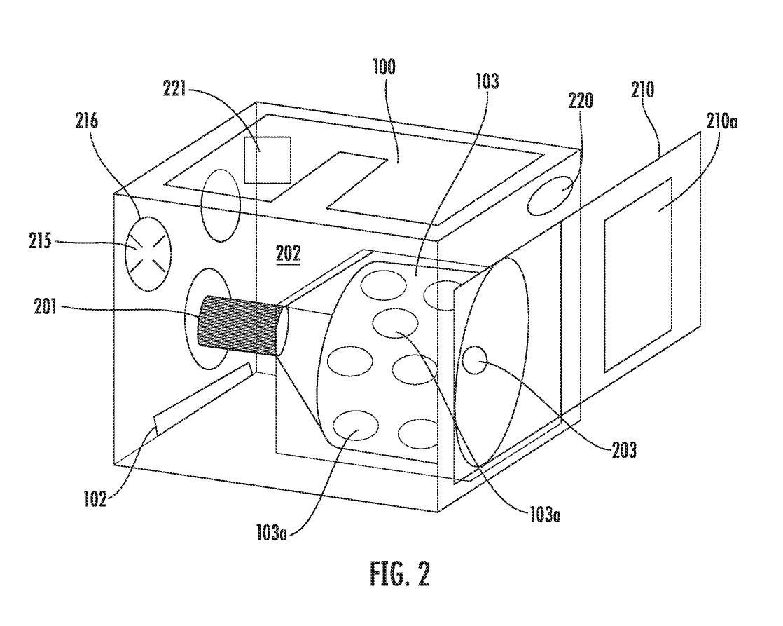

In FIG. 2, there is a view of the appliance with a tumbler of the present invention. In the appliance 100, there is the power supply 102, tumbler 103 with perforations 103a and cabinet interior 202. The holes are slits in the tumbler sized to minimize object tangles or loss. The lamp assembly 201 is shown as a single item but described in detail in FIG. 1. The tumbler 103 is shown wherein an object 203 is sitting directly in tumbler 103. In this view, the sealing door 210 is shown and can also have a window 210a. The door seals and can lock in place by means known in the art. Heat is vented from the lamp assembly 201 via exhaust 215 and exhaust fan 216. The control panel 220 has a circuit board 221 for controlling on/off, time, intensity, and the like, as taught herein. The interior walls of the cabinet interior 202 and walls of the inside of the tumbler have a reflective inactivation property/coating, reflecting high energy light from the lamp assembly 201. Polished stainless steel, aluminum, and thermal cured polymers are used in other embodiments. The walls are perforated just enough to allow for minimal thermal release. The following reflectivity is mainly for UVC while our systems includes DUV, MUV, and NUV. Reflectivity for: 1. ePTFE (expanded polytetrafluroethylene) 95% 2. Aluminum sputtered on glass 88% 3. Aluminum foil 73% and 4. Stainless steel 20-28%. Holes expect to be approximately 1/16 inch diameter sporadically to minimize reflectivity loss.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A sterilization appliance cabinet for sterilizing an object comprising:
a) a sealing door;
b) a cabinet interior having UV reflective walls and a ventilated tumbler for placement of the object;
c) a lamp assembly comprising a low heat, high energy UV lamp;
d) an appliance control panel; and
e) wherein the lamp assembly is positioned to deliver sufficient low heat, high intensity UV light to the cabinet interior tumbler to sterilize the object.

2. The sterilization appliance according to claim 1 wherein the lamp assembly includes a high intensity HgXe lamp.

3. The sterilization appliance according to claim 1 where the tumbler is cylindrical.

4. The sterilization appliance according to claim 1 wherein the control panel allows for the adjustment of exposure time.

5. The sterilization appliance according to claim 4 wherein the exposure time is between about 0.01 seconds to about 5.0 seconds.

6. The sterilization appliance according to claim 1 wherein the control panel allows for the adjustment of the lamp intensity.

7. The sterilization appliance according to claim 1 which further comprises a viewing window.

8. The sterilization appliance according to claim 1 wherein the sealing door locks during appliance use.

9. The sterilization appliance according to claim 1 wherein there is heat venting at the back of the cabinet.

10. The sterilization appliance according to claim 1 wherein the object comprises a heat sensitive material.

11. The sterilization appliance according to claim 10 wherein the object is a heat sensitive plastic.

12. The sterilization appliance according to claim 1 wherein the high energy Uv light is polychromatic.

13. The sterilization appliance according to claim 1 wherein the light passes through a light guide which delivers the light to the tumbler.

14. A method of sterilizing an object comprising:
a) placing the object to be sterilized in a tumbler of a sterilization appliance cabinet, the cabinet comprising:
i. a sealing door;
ii. a cabinet interior having reflective walls and a ventilated tumbler;
iii. a lamp assembly comprising a low heat, high energy UV lamp;
iv. a UV transparent tray designed to hold the object in the cabinet interior;
v. an appliance control panel; and
vi. wherein the lamp assembly is positioned to deliver a low heat UV polychromatic light to the cabinet interior; and
b) operating the appliance for a time sufficient to sterilize the object placed in the cabinet.

15. The method according to claim 14 wherein the object comprises a heat sensitive material.

16. The method according to claim 15 wherein the object is a heat sensitive plastic.

17. The method according to claim 15 wherein the high energy UV light is polychromatic.

18. The method according to claim 14 wherein the light passes through a light guide before reaching the tumbler.
